(19) United States
(12) Patent Application Publication
(10) Pub. No.: US 2023/0330696 A1
(43) Pub. Date: Oct. 19, 2023

(54) Method for dispensing a liquid, viscous, pasty, powdery, gaseous product by means of a dispensing device designed to be held and manipulated with one hand by a user

(71) Applicant: APOLLINA, Alixan (FR)

(72) Inventors: Pierre DUTHOIT, Malissard (FR); Jeanne DELALANDE, Valence (FR); Baptiste LAGRANGE, Valence (FR)

(21) Appl. No.: 17/720,811

(22) Filed: Apr. 14, 2022

(51) Int. Cl. B05C 17/01; B05C 17/005; B05C 11/10

(52) U.S. Cl. CPC B05C 17/0103; B05C 17/0052; B05C 17/00506; B05C 17/00523; B05C 11/1002; B05C 17/00593

(57) ABSTRACT

The method for dispensing of a liquid, viscous, pasty, powdery, or gaseous product by means of a dispensing device (100) adapted to being held and manipulated by a single hand of the user U comprises:
Taking a dispensing device (100);
Selecting a connection tip from a tip assortment A through G, each of them having a previously defined function;
Securing the tip chosen from the tip assortment A through G inside the dispensing device (100);
Selecting a mode among the different modes of the control program;
Positioning the dispensing device (100) in the work zone;
Activating a control button (400);
Releasing the control button (400);
Removing the selected tip from the dispensing device (100);

The present invention relates to a method for dispensing to enable the controlled diffusion of a liquid, viscous, pasty, powdery, or gaseous product by means of a dispensing device in which a connection tip is connected.

The method for dispensing according to the present invention is more particularly for the dispensing devices developed by the applicant, as described and protected in patent WO2019186072.

The dispensing device is composed of an elongated body adapted to being held and manipulated with a single hand by the user, and inside the device there is positioned a battery powering an electric motor or geared motor, allowing the driving of a thrust axis which is designed to cooperate with a connection tip taken from an assortment of connection tips.

Each connection tip comprises on the one hand means of fixation for its retention inside the dispensing device and on the other hand means of recognition which is activated when the tip is connected in said dispensing device, making it possible to initialize one of the control programs previously integrated in an electronic card.

The dispensing device comprises a command button enabling a control of the electric motor or geared motor depending on the control program as determined by the tip for the purpose of controlling the latter.

### BRIEF DESCRIPTION OF THE INVENTION

The method for dispensing according to the invention will describe and protect each step in the functioning of the dispensing device provided to the user depending on each connection tip taken from the assortment of tips.

The method for dispensing according to the present invention comprises:
Taking a dispensing device;
Selecting a connection tip from a tip assortment A through G, each of them having a previously defined function;
Securing the tip chosen from the tip assortment A through G inside the dispensing device so as to activate recognition means allowing a control program to be initiated, corresponding to the selected tip;
Selecting if necessary a mode among the different modes of the control program depending on the tip and by means of at least one command button;
Positioning the dispensing device in the work zone where the tip is located;
Activating a control button enabling the automatic advancement of a thrust piston of the dispensing device up to its initial position according to the previously determined control program;
Holding down the control button to start the control program according to the previously determined functions in order to control an electric motor driving the thrust piston housed in the dispensing device so as to act on the tip and the dispensing of said product;
Releasing the control button at the end of the application of the product contained in the tip;
Removing the selected tip from the dispensing device and/or activating a retraction button to control the electric motor driving the retraction of the thrust piston of the dispensing device from the tip.

The method for dispensing according to the present invention comprises:
Selecting and securing a tip from the assortment of type A inside the dispensing device so as to activate recognition means allowing the initialization of a control program corresponding to the selected tip;
Selecting one of the following modes of the control program depending on the determined by the user U:
   I. MA1
   II. MA2
   III. MA3

The method for dispensing according to the present invention comprises:
Selecting and securing a tip from the assortment of type B inside the dispensing device so as to activate recognition means allowing the initialization of a control program corresponding to the selected tip;
Activating a retraction button allowing the aspirating of a product contained in an external vial in order to fill the tip of type B;
Selecting one of the following modes of the control program depending on the work area as determined by the user U:
   I. MB1
   II. MB2
   III. MB3

The method for dispensing according to the present invention comprises:
Selecting and securing a tip from the assortment of type D inside the dispensing device so as to activate recognitions allowing the initialization of a control program corresponding to the selected tip;
Selecting one of the following modes of the control program depending on the work area as determined by the user:
   I. MD1
   II. MD2

The method for dispensing according to the present invention wherein mode MA1 of the control program allows the user U to select one of the following submodes regarding the movement of the thrust piston of the dispensing device:
Advancing the thrust piston at a constant speed Va10;
Advancing the thrust piston at a constant low speed Va11, then progressing from a speed Va11 to a constant elevated speed Va12;
Advancing the thrust piston at a constant elevated speed Va12;
Retraction of the thrust piston at a constant speed Vr.

The method for dispensing according to the present invention wherein mode MA2 of the control program allows the user U to select one of the following submodes regarding the movement of the thrust piston of the dispensing device (100):
Advancing the thrust piston at a constant speed Va17;
Advancing the thrust piston at a constant low speed Va13, then progressing from a low speed Va13 to a constant elevated speed Va14;
Advancing the thrust piston at a constant elevated speed Va14;
Retraction of the thrust piston at a constant speed Vr.

The method for dispensing according to the present invention wherein erode MA3 of the control program allows the user U to select one of the following submodes regarding the movement of the thrust piston of the dispensing device:
Advancing the thrust piston at a constant speed Va18;
Advancing the thrust piston at a constant low speed Va15, then progressing from a low speed Va15 to a constant elevated speed Va16;
Advancing the thrust piston at a constant elevated speed Va16;
Partial retraction of the thrust piston;
Retraction of the thrust, piston at a constant speed Vr.

The method for dispensing according to the present invention wherein mode MB1 of the control program allows the user U to select one of the following submodes regarding the movement of the thrust piston of the dispensing device:
Advancing the thrust piston at a constant speed Vb10;
Advancing the thrust piston at a constant low speed Vb11;
Advancing the thrust piston at a constant elevated speed Vb12;
Retraction of the thrust piston at a constant speed Vr.

The method for dispensing according to the present invention wherein mode MB2 of the control program allows the user U to select one of the following submodes regarding the movement of the thrust piston of the dispensing device:
Advancing the thrust piston at a constant speed Vb16;
Advancing the thrust piston at a constant low speed Vb13;
Advancing the thrust piston constant elevated speed Vb14;
Retraction of the thrust piston at a constant speed Vr.

The method for dispensing according to the present invention wherein mode MB3 of the control program allows the user U to select one of the following submodes regarding movement of the thrust piston of the dispensing device:
Advancing the thrust piston at a constant speed Vb15;
Retraction of the thrust piston at a constant speed Vr.

The method for dispensing according to the present invention comprises:
Securing a tip from the assortment of type C inside the dispensing device so as to activate recognition means allowing the initialization of a control program corresponding to the selected tip;
Selecting one of the following submodes for the movement of the thrust piston of the dispensing device:
   I. Advancing the thrust piston at a constant speed Vc10;
   II. Advancing the thrust piston at a constant low speed Vc1;
   III. Advancing the thrust piston at a constant elevated speed Vc2:
   IV. Retraction of the thrust piston at a constant speed Vr.

The method for dispensing according to the present invention determines that mode MD1 of the control program comprises:
Securing a tip from the assortment of type D inside the dispensing device so as to activate recognition means allowing the initialization of a control program corresponding to the selected tip;
Selecting one of the following submodes for the movement of the thrust piston of the dispensing device:
   I. Advancing the thrust piston at a constant speed Vd10;
   II. Advancing the thrust piston at a constant low speed Vd11;
   III. Advancing the thrust piston at a constant elevated speed Vd12;
   IV. Retraction of the thrust piston at a constant speed Vr.

The method for dispensing according to the present invention determines that mode MD2 of the control program comprises:
Securing a tip from the assortment of type D containing a very viscous liquid inside the dispensing device so as to activate recognition means allowing the initialization of a control program corresponding to the selected tip;
Selecting one of the following submodes for the movement of the thrust piston of the dispensing device:
   I. Advancing the thrust piston at a constant speed Vd20;
   II. Advancing the thrust piston at a constant low speed Vd21;
   III. Advancing the thrust piston at a constant elevated speed Vd22;
   IV. Retraction of the thrust piston at a constant speed Vr.

The method for dispensing according to the present invention comprises:
Securing a tip from the assort assortment of type E inside the dispensing device so as to activate recognition means allowing the initialization of a control program corresponding to the selected tip;
Selecting one of the following submodes for the movement of the thrust piston of the dispensing device:
   I. Advancing the thrust piston at a constant speed Ve10;
   II. Advancing the thrust piston at a constant low speed Ve11;
   III. Advancing the thrust piston at a constant elevated speed Ve12;
   IV. Retraction of the thrust piston at a constant speed Vr.

The method for dispensing according to the present invention comprises:
Securing a tip from the assortment of type F inside the dispensing device so as to activate recognition means allowing the initialization of a control program corresponding to the selected tip;
Selecting one of the following submodes for the movement of the thrust piston of the dispensing device:
   I. Advancing the thrust piston at a constant speed Vf10;
   II. Advancing the thrust piston at a constant low speed Vf11;
   III. Advancing the thrust piston at a constant elevated speed Vf12;
   IV. Retraction of the thrust piston at a constant speed Vr.

The method for dispensing according to the present invention comprises:
Securing a tip from the assortment of type G inside the dispensing device so as to activate recognition means allowing the initialization of a control program corresponding to the selected tip;
Selecting one of the following submodes for the movement of the thrust piston of the dispensing device:
   I. Advancing the thrust piston at a constant speed Vg10;
   II. Advancing the thrust piston at a constant low speed Vg11;
   III. Advancing the thrust piston at a constant elevated speed Vg12;
   IV. Retraction of the thrust piston at a constant speed Vr.

According to one preferred mode of the invention, the method for dispensing uses speeds Va through Vg and Vr which are between 0.05 millimeters per second and 15 millimeters per second.

The method for dispensing according to the present invention wherein the tips A, B, D and F can be previously heated.

According to another mode of the invention, the method for dispensing consists in that the tips A, B, D and F are heated on a recharging support prior to being secured in the dispensing device.

According to another mode of the invention, the method for dispensing consists in that the tips A, B, D and F are heated in the dispensing device.

According to another mode of the invention, the method for dispensing consists in that the tips A, B. D and F have their own heating device.

### BRIEF DESCRIPTION OF THE DRAWINGS

FIG. 1 is an exploded perspective view illustrating a dispensing device and a connection tip from an assortment of tips according to the present invention;

FIG. 2 is a perspective view showing the dispensing device assembled with the connection tip from an assortment of tips according to the present invention;

FIG. 3 is a perspective view representing the dispensing device and the connection tip arranged on a recharging support according to the present invention;

FIG. 4 is a perspective view illustrating the dispensing device joined to the tip, held and manipulated by a single hand of the user;

FIG. 5 is a schematic view showing the different steps of the method for dispensing implemented by the dispensing device according to the present invention;

FIGS. 6 and 7 are schematic views representing the different operating modes of the method for dispensing when the dispensing device is connected with a connection tip of type A or type B;

FIG. 8, 10 to 12 are schematic views illustrating the different operating modes of the method for dispensing when the dispensing device is connected with a connection tip of type C or type E or type F or type G;

FIG. 9 is a schematic view showing the different operating modes of the method for dispensing when the dispensing device is connected with a connection tip of type D;

FIG. 13 is a schematic view representing the different process steps of the method for dispensing implemented by the dispensing device according to the present invention.

### DESCRIPTION OF PREFERRED EMBODIMENTS

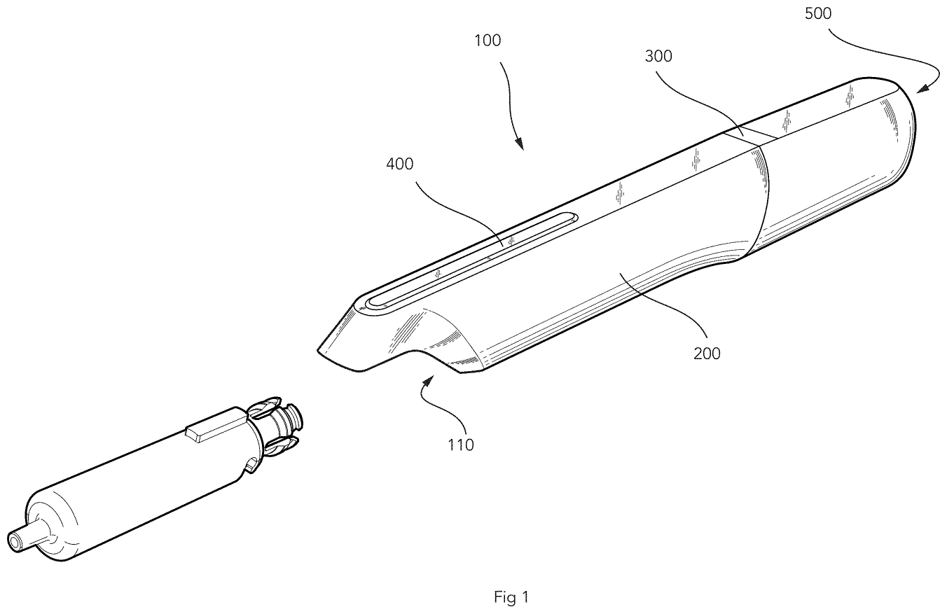

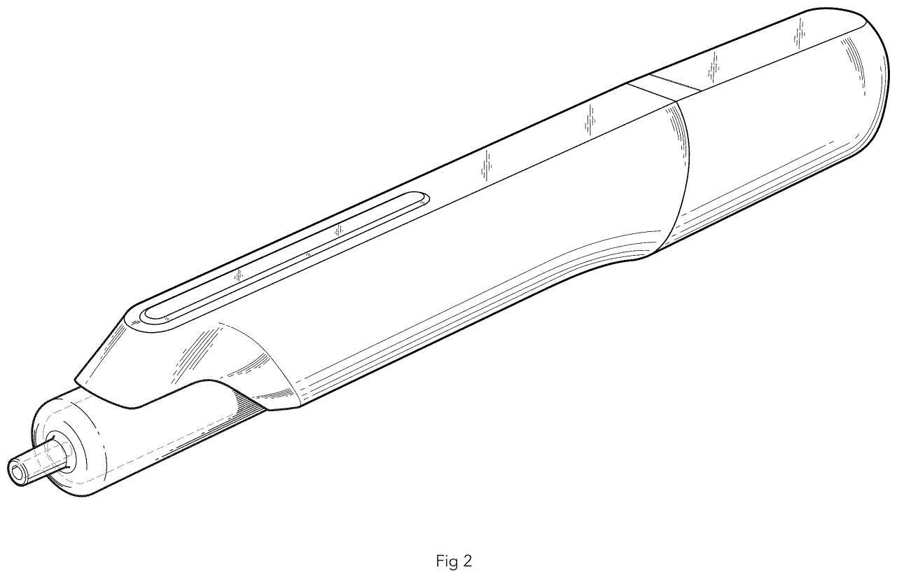

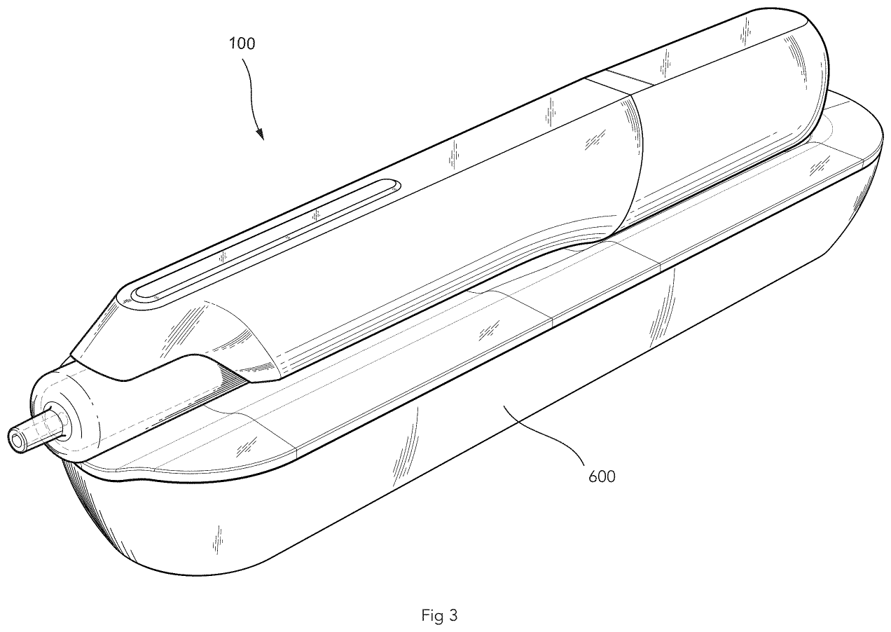

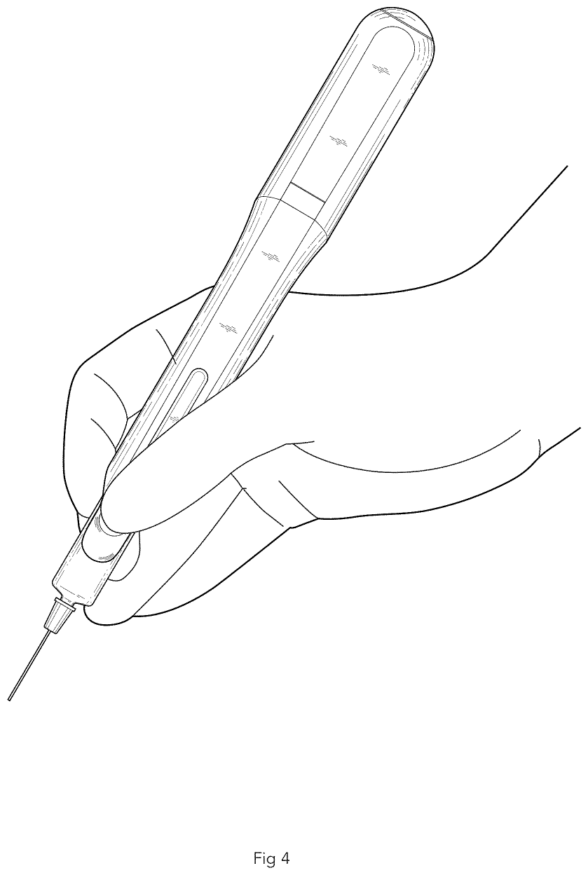

There is represented in FIGS. 1 to 4 a dispensing device 100 similar to that described and protected in the patent WO2019186072 belonging to the applicant.

The dispensing device 100 can be used in different fields and more particularly in the medical field, such as dentistry, orthopedics, cosmetics, ophthalmology and veterinary.

The dispensing device 100 comprises an elongated body 200 having a command button 300, a control button 400 and a retraction button 500 allowing the user U to select an operating mode previously recorded in an electronic control card, not illustrated, which is housed in said dispensing device.

The dispensing device 100 comprises at one of its ends, more particularly at the end opposite the retraction button 500, a housing 110 enabling the fixation of a connection tip taken from an assortment of tips A through G.

The connection tips A through G respectively make possible different procedures, namely:
I. Connection tip A allows the injecting of a dental anesthetic;
II. Connection tip B allows the filling and the dispensing of any type of liquid such as antiseptic for cleaning the dental canals during a root canal;
III. Connection tip C enables the dispensing of fluid from capsules of a powder/liquid mixture;
IV. Connection tip D allows the application of more or less viscous pastes from capsules;
V. Connection tip E allows the application of soft paste, such as that for molding a tooth impression;
VI. Connection tip F allows the application of any type of more or less viscous paste which is industrially prefilled, such as a dental composite;
VII. Connection tip G of a double syringe type allows the application of two pastes of any type, mixed by a mixer.

The dispensing device 100 comprises in its internal portion, as described in the patent WO2019186072 belonging to the applicant, recognition means allowing the initialization of a control program corresponding to the selected tip A through G, and an electric motor driving a thrust piston for acting on the tip which has been secured.

The dispensing device 100 can be placed on a recharging support 600 enabling a charging of the batteries provided inside said device, said batteries for example making it possible to drive the electric motor and power the electronic control card and the recognition means, allowing the initializing of a control program corresponding to the selected tip.

In our example as described below, the modes and submodes of the control program are the ones which are proposed in the field of dentistry.

Of course, and without this limiting the scope of the invention, these modes and submodes can be applied to other fields, such as but not limited to the medical fields already indicated above.

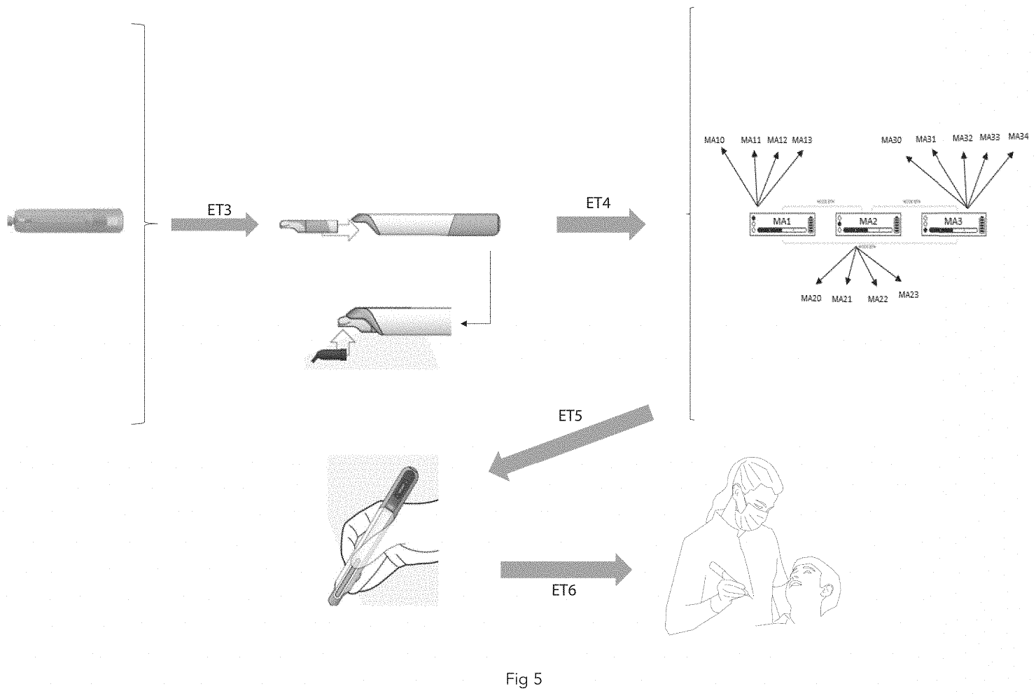

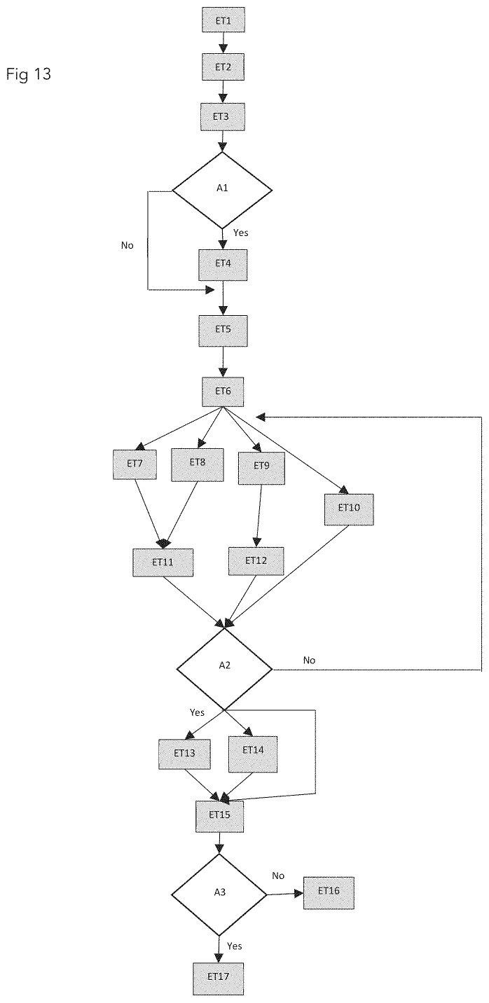

FIGS. 5 and 13 illustrate a diagram and a flow chart showing the different steps of the method for dispensing according to the present invention in order to implement the dispensing device 100 by a user U.

The first step ET1 involves the user U taking the dispensing device 100 from its recharging support 600 or wherever it may be located.

The second step ET2 allows the user U to select a connection tip, depending on the action to be carried out, from the assortment of tips A through G, each of them having a predefined function.

The third step ET3 allows the user U to put in place and secure the connection tip chosen from the tip assortment A through G inside the dispensing device 100. When the connection tip is secured inside the dispensing device 100, the latter can activate the recognition means housed in said dispensing device. The recognition means, of mechanical or electronic type, make it possible to initialize a control program loaded in the electronic control card corresponding to the selected tip A through G. The control program checks whether the selected tip A through G has various operating modes and/or submodes according to step A1 FIG. 13.

The fourth step ET4 allows the user U, once the control program has been initialized, to select, if necessary according to the function of the connection tip A through G and by using at least one command button 300, a particular operating mode among the various modes proposed by the control program.

The fifth step ET5 includes the user U positioning the dispensing device 100 in the work zone where the tip A through G should be located. In our nonlimiting exemplary embodiment, the user U will place the previously selected tip for example in the mouth of a patient P in order to perform the necessary treatment in the desired health care sequence (infiltration, irrigation, placement of reconstruction material, placement of impression paste, etc).

The sixth step ET6 allows the user U to actuate a control button 400 for the automatic advancement of a thrust piston driven by an electric motor housed inside the dispensing device 100 up to its initial position according to the previously determined control program.

According to the seventh step ET7, the user U holds down the control button 400, allowing the starting of the control program according to the previously determined functions, in order to drive the thrust piston housed in the dispensing device 100 so as to act on the tip A through G and distribute said product.

According to the eighth step ET8, the user U double clicks and holds down the control button 400 enabling the starting of the control program according to the previously determined functions, in order to drive the thrust piston housed in the dispensing device 100 so as to act on the tip A through G and distribute said product.

According to the ninth step ET9, the user U holds down the retraction button 500, enabling the starting of the control program according to the previously determined functions, in order to drive the thrust piston housed in the dispensing device 100 so as to act on the tip B.

According to the tenth step ET10, the user U double clicks on the retraction button 500, enabling the starting of the control program according to the previously determined functions, in order to drive the thrust piston housed in the dispensing device 100 so as to act on the tip B. During this step, the user U should verify that the procedure is terminated per A2 and move on to the following steps. If such is not the case, he may go back to the end of step ET6.

The eleventh step ET11 consists in the user U releasing the control button 400 at the end of the application of the product contained in the tip A through G. During this step, the user U should verify that the procedure is terminated per A2 and move on to the following steps. If such is not the case, he may go back to the end of step ET6.

The twelfth step ET12 consists in the user U releasing the retraction button 500 at the end of the aspiration of the product in the tip B. During this step, the user U should verify that the procedure is terminated per A2 and move on to the following steps. If such is not the case, he may go back to the end of step ET6.

During the thirteenth step ET13, the user U double clicks on a retraction button 500, enabling a control of the electric motor driving the retraction of the thrust piston of the dispensing device 100 from the tip.

During the fourteenth step ET14, the user U actuates a retraction button 500, enabling a control of the electric motor driving the retraction of the thrust piston of the dispensing device 100 from the tip.

During the fifteenth step ET15, the user U removes the selected tip A through G from the dispensing device 100.

The user U then proceeds with the verification steps per A3; depending on the tip A through G used, it will either be sent on for sterilization, step ET16, or disposed of, step ET17.

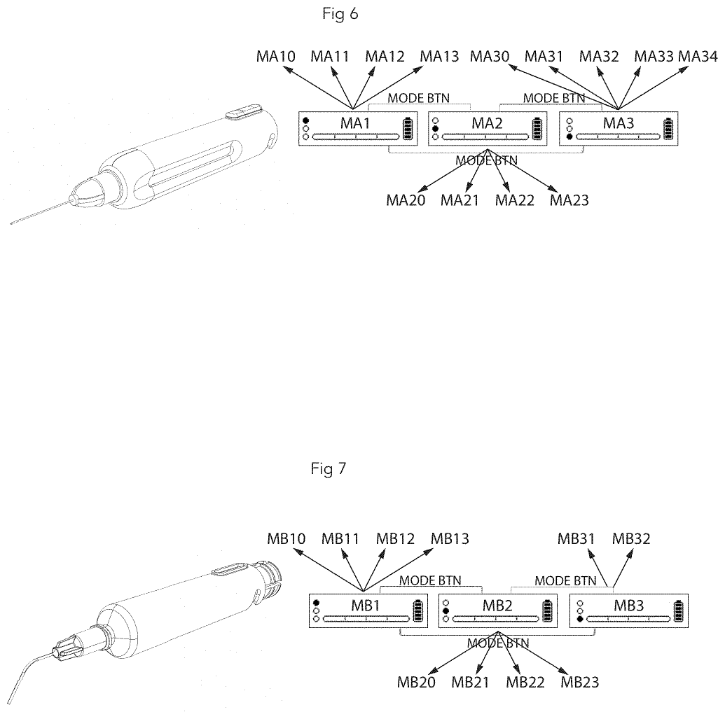

FIG. 6 represents the different operating modes of the method for dispensing when the user U connects a connection tip of type A to the dispensing device 100.

When the user U selects a connection tip A and this is secured inside the dispensing device 100, the recognition means is automatically activated, enabling the initializing of a control program corresponding to the selected tip.

Thus, the user U may select one of the following proposed modes in the control program through the control button 400:
I. MA1
II. MA2
III. MA3

In our example, namely, the field of dentistry, mode MA1 corresponds to a so-called “infiltration” mode. In this mode, the user U may select one of the following submodes:
MA10—this submode corresponds to the advancement of the thrust piston of the dispensing device 100 connected to the connection tip A at a constant speed Va10;
MA11—this submode corresponds to the advancing the thrust piston of the dispensing device 100 connected to the connection tip A at a constant low speed Va11, then progressing from a speed Va11 to a constant elevated speed Va12;
MA12—this submode corresponds to the advancing the thrust piston of the dispensing device 100 connected to the connection tip A at a constant elevated speed Va12;
MA13—this submode corresponds to the retraction of the thrust piston of the dispensing device 100 at a constant speed Vr.

Mode MA2 corresponds in the field of dentistry to a so-called “intra-ligamentary” mode. In this mode, the user U may select one of the following submodes:
MA20—this submode corresponds to the advancement of the thrust piston of the dispensing device 100 connected to the connection tip A at a constant speed Va17;
MA21—this submode corresponds to the advancing the thrust piston of the dispensing device 100 connected to the connection tip A at a constant low speed Va13, then progressing from a low speed Va13 to a constant elevated speed Va14;
MA22—this submode corresponds to the advancing the thrust piston of the dispensing device 100 connected to the connection tip A at a constant elevated speed Va14;
MA23—this submode corresponds to the retraction of the thrust piston of the dispensing device 100 at a constant speed Vr.

Mode MA3 corresponds in the field of dentistry to a so-called “truncular” mode. In this mode, the user U can select one of the following submodes:
MA30—this submode corresponds to the advancement of the thrust piston of the dispensing device 100 connected to the connection tip A at a constant speed Va18;
MA31—this submode corresponds to the advancing the thrust piston of the dispensing device 100 connected to the connection tip A at a constant low speed Va15, then progressing from a low speed Va15 to a constant elevated speed Va16;
MA32—this submode corresponds to the advancing the thrust piston of the dispensing device 100 connected to the connection tip A at a constant elevated speed Va16;
MA33—this submode corresponds to the retraction of the thrust piston of the dispensing device at a constant speed Vr.
MA34—this submode corresponds to the partial retraction of the thrust piston of the dispensing device.

It will be noted that the speeds Va10 through Va18 of each submode are between 0.05 millimeters per second and 15 millimeters per second. The constant speed Vr is between 0.05 millimeters per second and 15 millimeters per second.

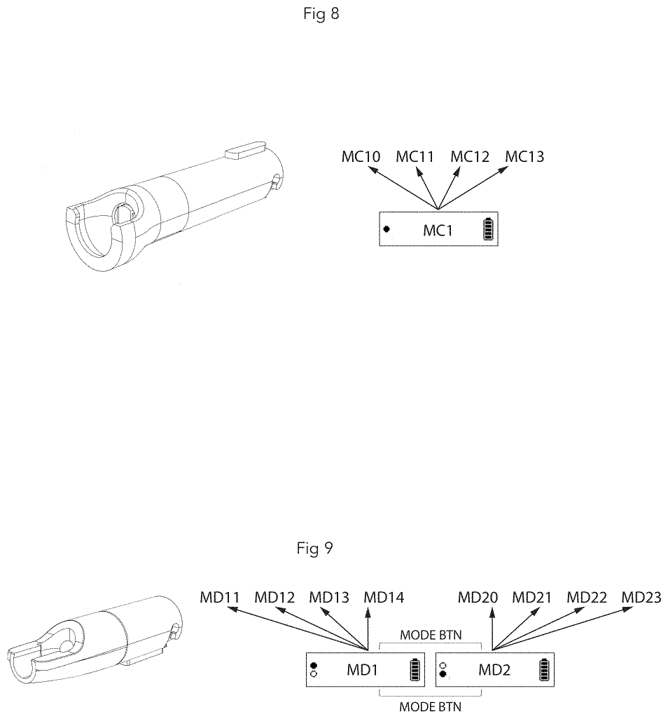

FIG. 7 shows the different operating modes of the method for dispensing when the user U connects a connection tip of type B to the dispensing device 100.

When the user U selects a connection tip B and this tip is secured inside the dispensing device 100, the recognition means is automatically activated to enable the initialization of a control program corresponding to the selected tip.

In a first step, the user activates the retraction button 500 allowing a product contained in an external bottle to be sucked up in order to fill the type B connection tip.

The user U can then use the control button 400 to select in the control program one of the following proposed modes:
I. MB1
II. MB2
III. MB3

In our example, namely, the field of dentistry, mode MB1 corresponds to a so-called “irrigation” mode of the control program. In this mode, the user U can select one of the following submodes:
MB10—this submode corresponds to the advancement of the thrust piston of the dispensing device 100 connected to the connection tip B at a constant speed Vb10;
MB11—this submode corresponds to the advancing the thrust piston of the dispensing device 100 connected to the connection tip B at a constant low speed Vb11;
MB12—this submode corresponds to the advancing the thrust piston of the dispensing device 100 connected to the connection tip B at a constant elevated speed Vb12;
MB13—this submode corresponds to the retraction of the thrust piston of the dispensing device at a constant speed Vr.

Mode MB2 corresponds in the field of dentistry to a so-called “purge” mode of the control program. This mode allows the user U to select one of the following submodes:
MB20—this submode corresponds to the advancement of the thrust piston of the dispensing device 100 connected to the connection tip B at a constant speed Vb16;
MB21—this submode corresponds to the advancing the thrust piston of the dispensing device 100 connected to the connection tip B at a constant low speed Vb13;
MB22—this submode corresponds to the advancing the thrust piston of the dispensing device 100 connected to the connection tip B at a constant elevated speed Vb14;
MB23—this submode corresponds to the retraction of the thrust piston of the dispensing device at a constant speed Vr.

Mode MB3 corresponds in the field of dentistry to a so-called “suction” mode of the control program. This mode allows the user to select one of the following submodes:
MB31—this submode corresponds to the advancing the thrust piston of the dispensing device 100 connected to the connection tip B at a constant elevated speed Vb15;
MB32—this submode corresponds to the retraction of the thrust piston of the dispensing device at a constant speed Vr.

It will be noted that the speeds Vb10 to Vb16 of each submode are between 0.05 millimeters per second and 15 millimeters per second. The constant speed Vr is between 0.05 millimeters per second and 15 millimeters per second.

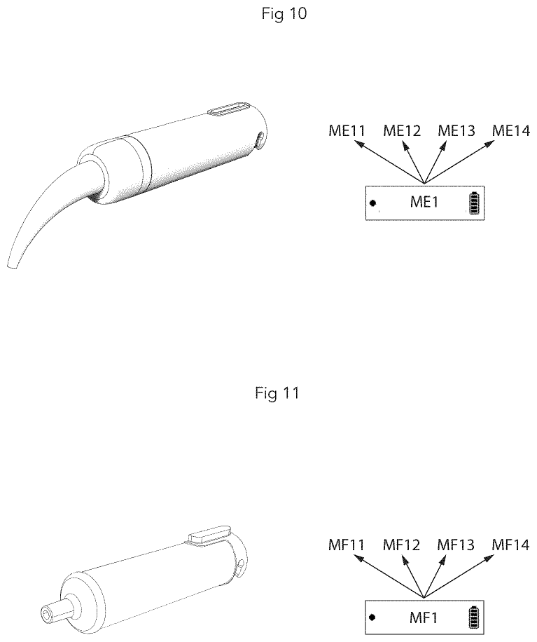

FIGS. 8 and 10 to 12 show the different operating modes of the method for dispensing when the dispensing device 100 is connected with a connection tip of type C or type E or type F or type G.

When the user U selects a connection tip C, E, F or G and this tip is secured inside the dispensing device 100, the recognition means is automatically activated to enable the initialization of a control program corresponding to the selected tip.

Thus, the user U can select with the control button 400 one of the modes MC1, ME1, MF1 or MG1 in the control program, depending on the selected tip, and can access one of the following submodes:
MC10, ME10, MF10 or MG10—this submode corresponds to the advancement of the thrust piston of the dispensing device 100 connected to the connection tip C, E, F or G at a constant speed Vc10, Ve10, Vf10, Vg10;
MC11, ME11, MF11 or MG11—this submode corresponds to the advancing the thrust piston of the dispensing device 100 connected to the connection tip C, E, F or G at a constant low speed Vc11, Ve11, Vf11, Vg11;
MC12, ME12, MF12 or MG12—this submode corresponds to the advancing the thrust piston of the dispensing device 100 connected to the connection tip C, E, F or G at a constant elevated speed Vc12, Ve12, Vf12, Vg12;
MC13, ME13, MF13 or MG13—this submode corresponds to the retraction of the thrust piston of the dispensing device 100 at a constant speed Vr.

It will be noted that the speeds Vc10, Ve10, Vf10, Vg10 to Vc12, Ve12, Vf12, Vg12 of each submode are between 0.05 millimeters per second and 15 millimeters per second. The constant speed Vr is between 0.05 millimeters per second and 15 millimeters per second.

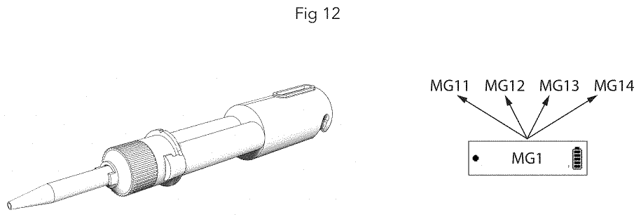

FIG. 9 shows the different operating modes of the method for dispensing when the dispensing device 100 is connected to a connection tip of type D.

When the user U selects a connection tip D and this tip is secured inside the dispensing device 100, the recognition means is automatically activated to enable the initialization of a control program corresponding to the selected tip.

Thus, the user U can use the control button 400 to select in the control program one of the following proposed modes:
MD1
MD2

In our example, namely, the field of dentistry, mode MD1 corresponds to a so-called “low viscosity liquid” mode. In this mode, the user U can select one of the following submodes:
MD10—this submode corresponds to the advancement of the thrust piston of the dispensing device 100 connected to the connection tip D at a constant speed Vd10;
MD11—this submode corresponds to the advancing the thrust piston of the dispensing device 100 connected to the connection tip D at a constant low speed Vd11;
MD12—this submode corresponds to the advancing the thrust piston of the dispensing device 100 connected to the connection tip D at a constant elevated speed Vd12;
MD13—this submode corresponds to the retraction of the thrust piston of the dispensing device 100 at a constant speed Vr.

Mode MD2 correspond in the field of dentistry to a so-called “high viscosity liquid” mode. In this mode, the user U can select one of the following submodes:
MD20—this submode corresponds to the advancement of the thrust piston of the dispensing device 100 connected to the connection tip D at a constant speed Vd20;
MD21—this submode corresponds to the advancing the thrust piston of the dispensing device 100 connected to the connection tip D at a constant low speed Vd21;
MD22—this submode corresponds to the advancing the thrust piston of the dispensing device 100 connected to the connection tip D at a constant elevated speed Vd22;
MBD23—this submode corresponds to the retraction of the thrust piston of the dispensing device 100 at a constant speed Vr.

It will be noted that the speeds Vd10 to Vd12 and Vd20 to Vd22 of each submode are between 0.05 millimeters per second and 15 millimeters per second. The constant speed Vr is between 0.05 millimeters per second and 15 millimeters per second.

It will be noted that the tips A, B, D and F can be previously heated in order to improve the efficacy of certain products contained in said tips.

In fact, the heating of dental composites resins improves their mechanical properties and thus increases their hardness, their adhesion, and the quality of the degree of conversion. The act of heating and maintaining the tips at an ideal temperature makes it possible to reduce the sensation of an injection, due to the sudden temperature difference, and thus to improve the comfort of the patient.

Likewise, the act of heating solutions for endodontics increases their solvent capability and thus increases the dissolution of organic and mineral materials by neutralizing microorganisms, thus improving the irrigation treatment.

The heating of the tips can be done by any known means, arranged either in the recharging support 600 or in the dispensing device 100 or in the connection tip itself.

It will be noted that the number of modes and submodes of the control program is not limited to the ones described above, but rather their number will be adapted according to the field in which the dispensing device 100 is used.

It will be understood that the devices just described and represented above have been done so with a view to a disclosure, and not a limitation. Of course, other arrangements, modifications and improvements might be made in the above examples without thereby leaving the scope of the invention.

1. A method for dispensing of a liquid, viscous, pasty, powdery, or gaseous product by means of a dispensing device (100) adapted to being held and manipulated by a single hand of the user U, characterized in that it comprises:
providing a dispensing device (100);
Selecting a connection tip from a tip assortment A through G, each of them having a previously defined function;
Securing the tip chosen from the tip assortment A through G inside the dispensing device (100) so as to activate recognition means allowing a control program to be initiated, corresponding to the selected tip;
Selecting if necessary a mode among the different modes of the control program depending on the tip and by means of a command button (300);
Positioning the dispensing device (100) in the work zone where the tip is located;
Activating a control button (400) enabling the automatic advancement of a thrust piston driven by an electric motor housed inside the dispensing device (100) up to its initial position according to the previously determined control program;
Pressing the control button (400) to start the control program according to the previously determined functions in order to drive the thrust piston housed in the dispensing device (100) so as to act on the tip and the dispensing of said product;
Releasing the control button (400) at the end of the application of the product contained in the tip;
Removing the selected tip from the dispensing device (100) and/or activating a retraction button (500) to control the electric motor driving the retraction of the thrust piston of the dispensing device (100) from the tip.

2. Method for dispensing according to claim 1, characterized in that it comprises:
Selecting and securing a tip from the assortment of type A inside the dispensing device (100) so as to activate recognition means allowing the initialization of a control program corresponding to the selected tip;
Selecting one of the following modes of the control program depending on the work area as determined by the user U:
I. MA1
II. MA2
III. MA3

3. Method for dispensing according to claim 1, characterized in that it comprises:
Selecting and securing a tip from the assortment of type B inside the dispensing device (100) so as to activate recognition means allowing the initialization of a control program corresponding to the selected tip;
Activating a retraction button allowing a product contained in an external bottle to be sucked up in order to fill the type B connection tip.
Selecting one of the following modes of the control program depending on the work area as determined by the user U:
I. MB1
II. MB2
III. MB3

4. Method for dispensing according to claim 1, characterized in that it comprises:
Selecting and securing a tip from the assortment of type D inside the dispensing device (100) so as to activate recognition means allowing the initialization of a control program corresponding to the selected tip;
Selecting one of the following modes of the control program depending on the work area as determined by the user U:
I. MD1
II. MD2

5. Method for dispensing according to claim 2, characterized in that mode MA1 of the control program allows the user U to select one of the following submodes regarding the movement of the thrust piston of the dispensing device (100):
Advancing the thrust piston at a constant speed Va10;
Advancing the thrust piston at a constant low speed Va11, then progressing from a speed Va11 to a constant elevated speed Va12;
Advancing the thrust piston at a constant elevated speed Va12;
Retraction of the thrust piston at a constant speed Vr.

6. Method for dispensing according to claim 2, characterized in that mode MA2 of the control program allows the user U to select one of the following submodes regarding the movement of the thrust piston of the dispensing device (100):
Advancing the thrust piston at a constant speed Va17;
Advancing the thrust piston at a constant low speed Va13, then progressing from a low speed Va13 to a constant elevated speed Va14;
Advancing the thrust piston at a constant elevated speed Va14;
Retraction of the thrust piston at a constant speed Vr.

7. Method for dispensing according to claim 2, characterized in that mode MA3 of the control program allows the user U to select one of the following submodes regarding the movement of the thrust piston of the dispensing device (100):
Advancing the thrust piston at a constant speed Va18;
Advancing the thrust piston at a constant low speed Va15, then progressing from a low speed Va15 to a constant elevated speed Va16;
Advancing the thrust piston at a constant elevated speed Va16;
Partial retraction of the thrust piston;
Retraction of the thrust piston at a constant speed Vr.

8. Method for dispensing according to claim 3, characterized in that mode MB1 of the control program allows the user U to select one of the following submodes regarding the movement of the thrust piston of the dispensing device (100):
Advancing the thrust piston at a constant speed Vb10;
Advancing the thrust piston at a constant low speed Vb11;
Advancing the thrust piston at a constant elevated speed Vb12;
Retraction of the thrust piston at a constant speed Vr.

9. Method for dispensing according to claim 3, characterized in that mode MB2 of the control program allows the user U to select one of the following submodes regarding the movement of the thrust piston of the dispensing device (100):
Advancing the thrust piston at a constant speed Vb16;
Advancing the thrust piston at a constant low speed Vb13;
Advancing the thrust piston at a constant elevated speed Vb14;
Retraction of the thrust piston at a constant speed Vr.

10. Method for dispensing according to claim 3, characterized in that mode MB3 of the control program allows the user U to select one of the following submodes regarding the movement of the thrust piston of the dispensing device (100):
Advancing the thrust piston at a constant speed Vb15;
Retraction of the thrust piston at a constant speed Vr.

11. Method for dispensing according to claim 1, characterized in that it comprises:
Securing a tip from the assortment of type C inside the dispensing device (100) so as to activate recognition means allowing the initialization of a control program corresponding to the selected tip;
Selecting one of the following submodes for the movement of the thrust piston of the dispensing device (100):
I. Advancing the thrust piston at a constant speed Vc10;
II. Advancing the thrust piston at a constant low speed Vc1;
III. Advancing the thrust piston at a constant elevated speed Vc2;
IV. Retraction of the thrust piston at a constant speed Vr.

12. Method for dispensing according to claim 4, characterized in that mode MD1 consists in:
Securing a tip from the assortment of type D inside the dispensing device (100) so as to activate recognition means allowing the initialization of a control program corresponding to the selected tip;
Selecting one of the following submodes for the movement of the thrust piston of the dispensing device (100):
I. Advancing the thrust piston at a constant speed Vd10;
II. Advancing the thrust piston at a constant low speed Vd11;
III. Advancing the thrust piston at a constant elevated speed Vd12;
IV. Retraction of the thrust piston at a constant speed Vr.

13. Method for dispensing according to claim 4, characterized in that mode MD2 consists in:
Securing a tip from the assortment of type D containing a very viscous liquid inside the dispensing device (100) so as to activate recognition means allowing the initialization of a control program corresponding to the selected tip;
Selecting one of the following submodes for the movement of the thrust piston of the dispensing device (100):
I. Advancing the thrust piston at a constant speed Vd20;
II. Advancing the thrust piston at a constant low speed Vd21;
III. Advancing the thrust piston at a constant elevated speed Vd22;
IV. Retraction of the thrust piston at a constant speed Vr.

14. Method for dispensing according to claim 1, characterized in that it comprises:
Securing a tip from the assortment of type E inside the dispensing device (100) so as to activate recognition means allowing the initialization of a control program corresponding to the selected tip;
Selecting one of the following submodes for the movement of the thrust piston of the dispensing device (100):
I. Advancing the thrust piston at a constant speed Ve10;
II. Advancing the thrust piston at a constant low speed Ve11;
III. Advancing the thrust piston at a constant elevated speed Ve12;
IV. Retraction of the thrust piston at a constant speed Vr.

15. Method for dispensing according to claim 1, characterized in that it comprises:
Securing a tip from the assortment of type F inside the dispensing device (100) so as to activate recognition means allowing the initialization of a control program corresponding to the selected tip;
Selecting one of the following submodes for the movement of the thrust piston of the dispensing device (100):
I. Advancing the thrust piston at a constant speed Vf10;
II. Advancing the thrust piston at a constant low speed Vf11;
III. Advancing the thrust piston at a constant elevated speed Vf12;
IV. Retraction of the thrust piston at a constant speed Vr.

16. Method for dispensing according to claim 1, characterized in that it comprises:
Securing a tip from the assortment of type G inside the dispensing device (100) so as to activate recognition means allowing the initialization of a control program corresponding to the selected tip;
Selecting one of the following submodes for the movement of the thrust piston of the dispensing device (100):
I. Advancing the thrust piston at a constant speed Vg10;
II. Advancing the thrust piston at a constant low speed Vg11;
III. Advancing the thrust piston at a constant elevated speed Vg12;
IV. Retraction of the thrust piston at a constant speed Vr.

17. Method for dispensing according to claim 5, characterized in that the speeds Va to Vg and Vr are comprised between 0.05 millimeters per second and 15 millimeters per second.

18. Method for dispensing according to claim 1, characterized in that the tips A, B, D and F can be previously heated.

19. Method for dispensing according to claim 18, characterized in that the tips A, B, D and F are heated on the recharging support prior to being secured in the dispensing device (100).

20. Method for dispensing according to claim 18, characterized in that the tips A, B, D and F are heated in the dispensing device (100).

21. Method for dispensing according to claim 18, characterized in that the tips A, B, D and F have their own heating device.
